(19) United States
(12) Patent Application Publication
(10) Pub. No.: US 2009/0306031 A1
(43) Pub. Date: Dec. 10, 2009

(54) 5alpha-ANDROSTANE-3,6,17-TRIONE (KNELLER'S TRIONE) AND METHODS OF USE THEREFOR

(76) Inventor: Bruce W. Kneller, Jackson, NJ (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/541,608

(22) Filed: Aug. 14, 2009

Related U.S. Application Data
(63) Continuation of application No. PCT/US2009/053148, filed on Aug. 7, 2009.

(51) Int. Cl. A61K 31/5685; C07J 1/00

(52) U.S. Cl. 514/177; 552/615

(57) ABSTRACT

Disclosed are compounds such as 5α-androstane-3,6,17-trione, formulations comprising said compounds, and methods of use thereof to stimulate endogenous testosterone production and improve athletic performance and libido.

### RELATED APPLICATIONS

This application is a continuation of International Application No. PCT/US2009/53148, filed Aug. 7, 2009. The entire teachings of this application is incorporated herein by reference.

### BACKGROUND OF THE INVENTION

Testosterone is the primary androgenic hormone responsible for secondary sexual characteristics in adult human males. Normal plasma levels of total testosterone in adult human males, which range from about 270 nanograms per deciliter (2,700 picograms per milliliter) to 1100 nanograms per deciliter (11,000 picograms per milliliter), are required for the full expression of the physical, psychological, and sexual characteristics of adult manhood. Estradiol, an estrogenic hormone, is the primary hormone responsible for the secondary sexual characteristics in female humans. Males, too, produce estradiol, and its presence in precise amounts is required for the activity of testosterone to be fully generated. Moreover, normal levels of estradiol, generally described as a plasma level of between about 10 picograms per milliliter and about 50 picograms per milliliter, are required for optimal bone density, cognitive function, cardiovascular health, and sexual function.

While individually the levels of plasma testosterone and plasma estradiol are of great importance to men, the ratio of plasma testosterone to plasma estradiol (T/E ratio) should also be within a certain range for a male to exhibit maximal, endocrinologic health and physical well being. Specifically, it is the presence of an abnormally low T/E ratio that is most problematic. If this ratio is too low, then estradiol related disorders like gynecomastia and excessive fat deposition may develop. Moreover, libido, lean body mass, athletic performance and intellectual ability may be diminished. Estradiol also increases the production of Sex Hormone Binding Globulin (SHBG), which then further reduces the quantity of free or biologically active testosterone available in the system. Additionally, a low T/E ratio has been associated with the increased risk of developing benign prostatic hypertrophy (BPH).

As men (i.e., human beings who are genetically male and who are at least 18 years of ages or who have otherwise clinically demonstrated to have ended the period of puberty) age, plasma testosterone level decreases. This is a factor that may contribute to some aspects of age-related physiological and psychological deterioration including but not limited to, decreased strength and muscle mass, decreased cognitive function and decreased libido. The decrease in testosterone production in older men often is not accompanied by a simultaneous decrease in estradiol production. Consequently, an undesirable T/E ratio is established. It is important to note that this decrease in not representative of an unhealthy increase in absolute estradiol levels, but rather due to a decrease in testosterone levels to the lower side of normal (on between about 270 nanograms per deciliter to on about 400 nanograms per deciliter or on between about 2,700 picograms per milliliter to on about 4,000 picograms per milliliter) or below normal (less than 270 nanograms per deciliter or less than 2,700 picograms per milliliter) range.

Although androgen (i.e., testosterone) replacement therapy has been shown to have beneficial effects in frankly hypogonadal men, its use in elderly men with borderline hypogonadism is somewhat controversial. Furthermore, current testosterone replacement methods have important limitations. Oral androgens are potentially hepatotoxic and injectable testosterone esters result in supraphysiological peaks of testosterone followed by hypogonadal troughs. Transdermal testosterone patches frequently cause local skin reactions and are associated with a decline in plasma testosterone concentration towards the end of the treatment period. Testosterone gels appear to cause fewer dermatological reactions but are often messy and can be associated with the undesirable transmission of testosterone from male patients to female partners. All exogenous androgens, including transdermals, lead to shut down of the Hypothalamic Pituitary Testiscular Axis (HPTA) and consequently, induce testicular atrophy and reduce fertility. Furthermore, exogenous androgens do not necessarily ameliorate the abnormally high T/E ratio seen in many older, hypogonadal males. The exogenous androgens may still be converted to various estrogens including estradiol by aromatization via a Cytochrome P-450 enzyme. Aromatase is an enzyme that catalyzes three consecutive hydroxylation reactions, converting C19 androgens (e.g., testosterone) to C18 estrogenic steroids (e.g., estradiol). On receiving electrons from NADPH-cytochrome P-450 reductase, aromatase converts testosterone to estradiol and androstenedione to estrone. Further, gynecomastia is a common side effect of all the above listed methods of androgen administration.

While it may be advantageous to increase plasma testosterone and androgen levels in various populations of men, a dramatic reduction of peripheral estrogen levels is not desirable; specifically it is undesirable to reduce estrogen levels to below accepted normal limits. Below normal levels of estrogens are well known to be associated with a variety of significant pathologies including, but not limited to, osteopenia, cardiovascular disease, blood lipid disorders, coagulation irregularities, libido issues and psychological problems. Thus, a well tolerated orally administered agent without these problems and side effects may have unique potential as a means of androgen replacement therapy.

### SUMMARY OF THE INVENTION

Disclosed are compounds such as 5α-androstane-3,6,17-trione (Kneller's Trione), formulations comprising said compounds, and methods of use thereof to stimulate endogenous testosterone production and improve athletic performance and libido. In certain embodiments the compounds and formulations of the invention increase endogenous testosterone in adult human males without altering estradiol levels from normal ranges. In certain embodiments the compounds and formulations of the invention increase the testosterone/estradiol (T/E) ratio.

It is an object of the invention to provide a method of increasing plasma testosterone level while preventing (i.e., without inducing) changes in plasma estradiol levels above or below accepted, laboratory normal limits. The administration of Kneller's Trione has been found to be effective and successful in this regard. Other objects of this invention include, but are not limited to, increasing lean muscle mass, increasing athletic performance and increasing libido in men through increasing plasma testosterone levels in men and/or increasing the plasma testosterone:estradiol ratio (T/E ratio) in men.

In one embodiment, the invention relates to a method of increasing endogenous testosterone production in a male comprising administering an effective amount of 5α-androstane-3,6,17-trione (Kneller's Trione) to said male. In one embodiment the increase in endogenous testosterone production results in an absolute increase in the testosterone-to-estrogen ratio (T/E). In certain embodiments, 5α-androstane-3,6,17-trione (Kneller's Trione) is administered orally, transdermally, parenterally, buccally, sublingually, or rectally. In particular embodiments the effective amount of Kneller's Trione is from about 25 mg to about 750 mg per day, more preferably from about 25 mg to about 500 mg per day, more preferably from about 25 mg to about 250 mg per day, and even more preferably from about 25 mg to about 100 mg per day.

In certain embodiments the increase in testosterone or increase in T/E ratio results in an increase in one or more of muscle mass, libido and athletic performance. Accordingly the invention also provides methods of increasing one or more of muscle mass, libido and athletic performance comprising administering an effective amount of Kneller's Trione to an individual, particularly to a human male. In particular embodiments the effective amount of Kneller's Trione is greater than 10 mg, preferably from about 25 mg to about 750 mg per day, more preferably from about 25 mg to about 500 mg per day, more preferably from about 25 mg to about 250 mg per day, and even more preferably from about 25 mg to about 100 mg per day.

The invention also relates to formulations comprising 5α-androstane-3,6,17-trione (Kneller's Trione) in an amount effective to increase endogenous testosterone production and/or T/E ratio in a male to whom the formulation is administered. In certain embodiments the formulation comprises 5α-androstane-3,6,17-trione (Kneller's Trione) in an amount sufficient to deliver from about 25 to about 750 mg per day to a user. In some embodiments the formulation is suitable for oral delivery. In other embodiments the formulation is a dietary supplement. The invention also relates to a formulation comprising from about 2.5% to about 100%, preferably from about 10% to about 100%, more preferably from about 25% to about 100%, and most preferably from about 50% to about 100% 5α-androstane-3,6,17-trione (Kneller's Trione).

### BRIEF DESCRIPTION OF THE DRAWING

FIG. 1 shows the structure and basic facts relating to 5α-androstane-3,6,17-trione (Kneller's Trione).

### DETAILED DESCRIPTION OF THE INVENTION

It is known that the HPTA is regulated by both androgens and estrogens, specifically and including, estradiol in men; the more powerful regulator, however, is estradiol. In men, plasma testosterone is extensively metabolized via aromatization to estradiol at the hypothalamus, and this estradiol is then immediately available to bind to hypothalamic estradiol receptors. The end result is a decrease in the secretion of Gonadotropin Releasing Hormone (GnRH). GnRH is a hormone that is responsible for signaling the pituitary gland to release gonadotropins—specifically Luteinizing Hormone (LH) and Follicle Stimulating Hormone (FSH). LH and FSH are then released into systemic circulation, where they travel to the testicles and stimulate the production of testosterone and spermatazoa.

It is also known than the administration of anti-estrogenic drugs to males causes an increase in the production of testosterone by interfering with the normal, estradiol-mitigated negative feedback loop that starts at the hypothalamus (J Clin Endocrinol Metab 91:1646-1653).

Some anti-estrogenic drug's effects on androgen production are likely directly mediated by a reduction in estradiol production. By reducing estradiol synthesis and, hence, estradiol's negative feedback on the pituitary and hypothalamus, some anti-estrogenic drugs appear to stimulate pituitary LH secretion sufficiently to increase endogenous testosterone production significantly in men. Furthermore, this increase in gonadrotropin secretion is likely responsible for limiting the reduction of estradiol levels and maintaining normal plasma estradiol levels. In support of this idea, it has been previously demonstrated that GnRH-analog-blocked, but testosterone replaced men have much lower plasma estradiol levels when given similar doses of an anti-estrogenic drug (J Clin Endocrinol Metab 88:204-210).

Whereas it is well know by those skilled in the art that gonadal steroid levels decrease as men age, the mechanism underlying this decrease has not been well defined. In fact, unless testosterone levels are severely depressed or other hormonal abnormalities coexist, the exact etiology of the hypogonadism is rarely related to overt gonadal or pituitary pathology (J Urol 155:529-533). This observation suggests that more subtle physiological alterations are responsible for the changes in testosterone level observed in aging men. For example, it has been suggested that the increase in gonadrotropin levels in aging men is a result of Leydig cell resistance to pituitary stimulation (J Clin Endocrinol Metab 73:1016-1025). Also, it has been suggested that the gonadotropin-suppressive activity of testosterone is increased as men age (Metabolism 33:1052-1059). Finally, as aromatase activity increases with age, an alteration in the T/E ratio may contribute to decreased androgen production (World J Urol 20:23-27). The effects of specific anti-estrogenic drugs known as aromatase inhibitors are consistent with a role of increased aromatization in testosterone deficiency in aging men (J Clin Endocrinol Metab 89:1174-1180).

There are currently two classes of anti-estrogenic drugs. The first class is estradiol receptor antagonists (ERA's). Examples of these are the drugs include, but are not limited to tamoxifen, toremifene, raloxifene, proloxifene, lasoxifene, idoxifene, arzoxifene, bazodoxifene and clomiphene. Drugs in this class bind to the estradiol receptor (ER) but do not cause activation of estradiol-responsive genes like normal estrogens do. Drugs in this class compete with endogenous estradiol and estrone at the receptor and block out the activity of endogenous estrogens. These drugs do, however, have a certain degree of pro-estrogenic activity and therefore may act like endogenous estrogens in certain tissues. Thus they are not purely anti-estrogenic or anti-estradiolic.

Furthermore, ERA's lead to an absolute increase in the levels of systemic and plasma estradiol due to their stimulatory effects on the production of androgens—which are metabolized to estradiol and estrone via Cytochrome P-450 aromatase. As a result, there can be a significant increase in estrogenic effects when and if the ERA's are discontinued.

The other class of anti-estrogenic drugs are known as Aromatase Inhibitors (AI's). Collective, non-limiting examples of drugs in this class are testolactone, aminoglutethimide, letrozole, anastrazole, finrazole, fadrozole, vorozole, formestane, atamestane, and exemestane. These drugs work by blocking and inactivating the aromatase enzyme. The aromatase enzyme is responsible for the formation of estrogens such as estradiol and estrone from androgenic precursors (i.e., testosterone and androstenedione). Aromatase inhibitors, therefore, prevent the actual formation of estrogens in the body, as opposed to ERA's which merely block the activity of circulating estrogens such as estradiol and estrone. Since aromatase inhibitors do not possess any sort of incidental, pro-estrogenic activity like ERA's, they are more preferred.

Aromatase inhibitors can be divided into two main subtypes. These subtypes are the (1) triazole based AI's and (2) the steroidal based AI's. Triazole based AI's (i.e., anastrazole, letrozole, fadrozole, vorozole and finrazole, etc.) have a triazole functional group that interacts with the heme prosthetic group of the aromatase enzyme and act as competitive inhibitors with respect to androgen substrates. Steroidal AI's (i.e., exemestane, formestane, and atamestane, etc.) work as substrate analogs that compete with the substrate for the aromatase enzyme. Steroidal AI's are also mechanism-based inhibitors, which require the catalytic ability of active aromatase enzyme to convert them to active intermediates. These active intermediates then bind irreversibly to the enzyme and cause its inactivation in a time-dependent manner. The development of AI's and some of their applications have been reviewed recently by Bruggemeier et al., Endocr Rev 26:331-345.

The use of aromatase inhibitors, preferably steroidal or mechanism-based aromatase inhibitors, prevents estrogenic rebound upon discontinuation since systemic and plasma estrogen levels, specifically, systemic and plasma estradiol level, are not elevated as may occur with the use of ERA's. There simply is a gradual return to baseline homeostatic sex hormone levels that were present before therapy was initiated.

Of the two subtypes of AI's (triazole and steroidal) steroidal AI's provide significant benefit if the goal is to raise plasma testosterone level, T/E ratio and/or reduce estrogenic rebound upon cessation or interruption of therapy. The reasons for this are that it has been shown that some triazole based AI's (e.g., letrozole and anastrazole) have been shown to actually increase aromatase enzyme levels, while other steroidal based AI's (e.g., exemestane) have been shown to actually decrease levels of aromatase enzyme. Steroidal AI's destabilize the aromatase enzyme and likely cause the involvement of a protease to degrade the aromatase enzyme (Cancer Res (66)21:10281-10286). Formestane and the steroidal AI, androst-1,4,6-triene-3,17-dione, have also been shown to decrease aromatase stability in quail brains (Brain Res 701:267-278). Since it is well known by those skilled in the art that aromatase activity in men increases with age, steroidal based AI's may offer a substantial and tangible benefit over triazole based AI's due to the steroidal AI's purported ability to destabilize the aromatase enzyme and decrease the systemic and plasma levels of such.

The idea of utilizing an AI to increase testosterone levels in men is not new; however, peripheral estrogen reduction is typically intense with AI use in men. Thus, the improvements in T/E ratio seen with the use of AI's may be relative in nature and are not necessarily absolute. Similar reductions of peripheral estrogen levels with the use of nonsteroidal AI's in men are well documented and are well known by those skilled in the art.

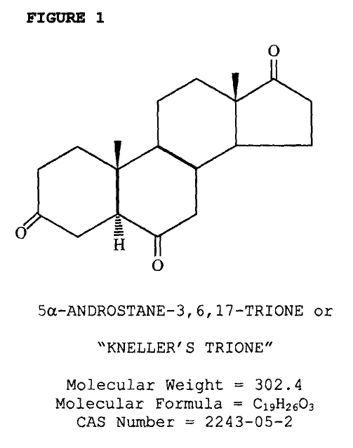

The invention relates to Kneller's Trione (5α-androstane-3,6,17-trione, CAS# 2243-05-2, Molecular Weight of about 302.4, Molecular Formula of C₁₉H₂₆O₃; FIG. 1), to formulations comprising Kneller's Trione, and to methods of using such compounds and formulations. During the course of research on the effect of Kneller's Trione on the sex hormone profile in men, it was surprisingly discovered that Kneller's Trione produced substantial and significant increases in plasma testosterone levels in men while inducing no changes outside the accepted normal limits for plasma estradiol levels. This indicates that an absolute improvement in the T/E ratio was achieved, as plasma estradiol levels were still maintained within the accepted normal limits while testosterone levels greatly increased. Thus, Kneller's Trione represents a significant improvement in the art when compared to many other AI's as a means of absolute increase of the T/E ratio. Specifically, Kneller's Trione does not adversely affect endogenous estradiol production in men, while many other AI's tend to suppress endogenous estradiol production in men to below accepted normal limits.

Equally surprising, work described herein revealed that the oral administration of Kneller's Trione is capable of increasing plasma free testosterone levels by more than 100%. Blood testosterone levels as a surrogate marker for muscle are well known (J Clin Endocrinol Metab 91:1646-1653, 2006). Thus, increases in blood testosterone levels within and above the normal range for males, however produced, can be confidently predicted to have significant ergogenic effects. It is well known that testosterone's effects on muscle mass, strength, and athletic performance are proportional to blood levels attained. This same study (J Clin Endocrinol Metab 91:1646-1653, 2006) predicts that effective estrogen blockers would produce significant and sustained elevations of blood testosterone concentrations and likely myotrophic and ergogenic effects in men treated with such drugs. Thus, an effective dose of Kneller's Trione can be used to facilitate increases muscle size, strength, and athletic performance in men via an increase in testosterone level both within the normal range and into supraphysiological range. The composition and formulations can have one or more (combinations or subcombinations) of the listed physiological effects.

Accordingly, the invention relates to compositions and formulations (e.g., drug, nutritional supplement, food supplement, dietary supplement) comprising, consisting essentially of, or consisting of Kneller's Trione, and to methods of making and using said compositions and formulations. As used herein, a nutritional supplement, also known as food supplement or dietary supplement, is a preparation for supplying effective agents that are missing or are not consumed in sufficient quantity in an individual's diet to have a desired effect. Typically the nutritional supplement is orally ingested.

Kneller's Trione can be made by any suitable method. For example, exemplary (non-limiting) embodiments of methods of making Kneller's Trione from bacterial transformation of androstenedione have been well described by Schaaf and Dettner (J Steroid Biochem Molec Biol 67(5-6):451-465) and Al-Awadi and Oomen (Steroids 70:327-333).

The invention also relates to a method of increasing endogenous testosterone production in a male comprising administering an effective amount of 5α-androstane-3,6,17-trione (Kneller's Trione) to said male. This increase in endogenous testosterone production may result in improvements in athletic performance, muscle mass and/or libido.

Kneller's Trione may be administered with or without a high protein diet, anaerobic training program or other dietary or training regimens in order to increase endogenous testosterone production, thereby increasing the variables associated with athletic function for the purpose of enhancing athletic performance. The oral, daily dose of Kneller's Trione can be from about 25 mg to about 750 mg per day, such as, for example, from about 25 mg to about 500 mg per day, from about 25 mg to about 250 mg per day, or from about 25 to about 100 mg per day. The daily dosage may be administered as a single daily dose or may be distributed among multiple daily doses. As used herein, the term “about” refers to a +/−10% variation from the nominal value. It is to be understood that such a variation is always included in any given value provided herein, whether or not it is specifically referred to.

The invention also relates to formulations comprising Kneller's Trione, including, but not limited to, dietary or nutritional supplement formulations. In particular embodiments, the composition or formulation comprises Kneller's Trione in an amount of from about 2.5% to about 100%, preferably from about 10% to about 100%, more preferably from about 25% to about 100%, and most preferably from about 50% to about 100% by weight of the composition or formulation. In certain embodiments, the composition comprises additional active ingredients and/or is formulated for oral use.

In some embodiments of the invention the composition comprising Kneller's Trione is formulated as a tablet, capsule, caplet, powder, suspension, gel preparation, aqueous solution, solid food form (e.g., chewable bar or wafer), or liquid dosage form such as elixirs, syrups, dispersed powders, granules or emulsions. In one embodiment the composition is particularly formulated for oral use. Although working examples described herein focused on the oral administration of Kneller's Trione in men, it is clear that compositions and formulations of the invention may be administered effectively by other routes including, but not limited to, parenteral, buccal, sublingual, rectal, and transdermal. In preferred embodiments the route of administration is oral, and suitable means are especially tablets, caplets, capules, pulls, suspensions, solutions, elixirs that can be produced in ways that are commonly used and familiar to one skilled in the art, with the additives and vehicles that are commonly used. As non-limiting examples, extended release or time release formulations are among technologies known to the skilled artisan and suitable for use with the invention.

In addition, Kneller's Trione can be administered or formulated alone or can be formulated with or administered before, concurrent with or after other optional components such as other active ingredients. In some embodiments the composition or formulation comprising Kneller's Trione contains one or more of the following ingredients, preferably as an active ingredient:
Carbohydrates including, but not limited to, isomaltulose, trehalose, maltodextrin, glucose, sucrose, fructose, lactose, amylose, and/or ribose;
Water soluble vitamins including, but not limited to, Vitamin C, Vitamin B1, Vitamin B2, Vitamin B3, Vitamin B6, Vitamin B12, and/or Vitamin K (and derivatives);
Minerals including, but not limited to, calcium, sodium, potassium, chromium, vanadium, magnesium, and/or iron (and derivatives) (preferably in amounts less than the RDA);
Amino acids including, but not limited to, L-arginine, L-ornithine, L-glutamine, L-tyrosine, L-taurine, L-leucine, L-isoleucine, and/or L-valine (and derivatives);
Nutraceutically acceptable stimulants including, but not limited to, methylxanthines (e.g.—caffeine) and/or glucuronolactone (and derivatives);
Nutraceutically acceptable hypoglycemic agents including, but not limited to, alpha-lipoic acid and its derivatives, cinnamon bark, bitter melon extracts, Gymnema Sylvestre extracts, 4-hydroxy-isoleucine, corosolic acid, and/or D-pinitol (and derivatives);
Creatine and its salts (e.g., creatine monohydrate), esters (e.g., creatine ethyl ester), chelates, amides, ethers (and derivatives);
Adenosine triphosphate and its disodium salt;
Glycerol and glycerol monostearate;
Mannitol;
Sorbitol; and
Dextran.

The compositions may contain pharmaceutically, e.g., nutraceutically, acceptable excipients, according to methods and procedures well known in the art. As used herein, “excipient” refers to substances that are typically of little or no therapeutic value, but are useful in the manufacture and compounding of various pharmaceutical preparations and which generally form the medium of the composition. These substances include, but are not limited to, coloring, flavoring, and diluting agents; emulsifying, dispersing and suspending agents, ointments, bases, pharmaceutical solvents; antioxidants and preservatives; and miscellaneous agents. Suitable excipients are described, for example, in Remington's Pharmaceutical Sciences, which is incorporated herein by reference in its entirety.

The compositions and formulations according to the present invention can further comprise one or more acceptable carriers. A wide number of acceptable carriers are known in the nutritional supplement arts, and the carrier can be any suitable carrier. The carrier need only be suitable for administration to animals, including humans, and be able to act as a carrier without substantially affecting the desired activity of the composition. Also, the carrier(s) may be selected based upon the desired administration route and dosage form of the composition. For example, the nutritional supplement compositions according to the present invention are suitable for use in a variety of dosage forms, such as liquid form and solid form (e.g., a chewable bar or wafer). In desirable embodiments the compositions and formulations comprise a solid dosage form, such as a tablet or capsule. Examples of suitable carriers for use in tablet and capsule compositions include, but are not limited to, organic and inorganic inert carrier materials such as gelatin, starch, magnesium stearate, talc, gums, silicon dioxide, stearic acid, cellulose, and the like. Desirably, the carrier is substantially inert, but it should be noted that the nutritional supplement compositions of the present invention may contain further active ingredients in addition to creatine bicarbonate.

The invention also relates to a method of increasing athletic performance in a male comprising administering to the male a composition or formulation comprising Kneller's Trione. The compositions and methods of the present invention may provide significant increase or improvement in athletic performance, e.g., muscle size, and/or muscle strength, and/or muscle endurance in individuals. As used herein, “athletic performance” and/or “athletic functions” refers to the sum of physical attributes which can be dependent to any degree on skeletal muscle contraction. For example, athletic performance and/or athletic functions include, but are not limited to, maximal muscle power, muscular endurance, running speed and endurance, swimming speed and endurance, throwing power, lifting and pulling power. Athletic performance can be measured by measuring any of a number of parameters, including, but not limited to, improvement in maximal power/strength, work performed during exercised of maximal effort muscle contractions, single effort sprint performance, and work performed during repetitive sprint performance.

While it is expected that the compositions and methods of the present invention will be of particular importance to bodybuilders and other athletes, the usefulness of compositions and methods of the invention is not limited to those groups. Rather, any individual in whom it is desirable to increase the T/E ratio may beneficially use the compositions and methods of the invention. Indeed, the disclosed compositions and methods have application to all animals, including mammals, birds and reptiles. As used herein, the term “animal” includes all members of the animal kingdom, preferably mammals (e.g., dogs, horses, cows, mules), more preferably humans. For example, the compositions and formulations of the invention may have beneficial effect for competitive animals (e.g., racehorses, show horses, racing dogs (e.g., greyhounds), bird dogs, show dogs) and work animals (e.g., horses, mules and the like) in whom an increase in muscle performance is desirable.

In a preferred embodiment compositions and formulations of the invention are administered to a male, particularly a human man. In certain embodiments the male may be an elderly male or a male having deficient (below normal) testosterone levels. In other embodiments the male does not have deficient testosterone levels.

The compositions and formulations according to the present invention may be employed in methods for supplementing the diet of an individual, e.g., a male, an athlete, etc., and/or for enhancing an individual's muscle mass and/or muscle size and/or strength, and/or endurance. Accordingly, the present invention provides methods of supplementing the dietary intake of an individual comprising administering to the individual an effective amount of a composition or formulation (e.g., Kneller's Trione or a composition comprising Kneller's Trione) according to the present invention to increase athletic performance or athletic function is said individual. The invention also relates to methods of improving athletic performance and/or athletic function in an individual comprising administering an effective amount of Kneller's Trione (alone or in combination with other agents) to the individual.

As used herein, an “effective amount” of compositions of the present invention is defined as an amount effective, at dosages and for periods of time necessary, to achieve the desired result. The effective amount of compositions of the invention may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present invention may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day, depending on the exigencies of the situation.

The embodiments set forth in the present application are provided only to illustrate various aspects of the invention and additional embodiments and advantages of the food supplements and methods of the present invention will be apparent to those skilled in the art. The teachings of all references and documents cited herein are incorporated herein by reference in their entirety.

The invention will be further illustrated by the following non-limiting examples.

### EXAMPLES

A pilot study conducted at the Ohio Research Group in Wadsworth, Ohio 44281, USA at the inventor's direction utilizing oral daily dosages of Kneller's Trione ranging from 25 mg to 750 mg revealed the following data.

The first study subject was a man, aged 23, who was dosed once per day orally with 25 mg per day of Kneller's Trione. Table 1 summarizes the data obtained.

TABLE 1
25 mg per day, single dose, Kneller's Trione

| | TOTAL | BIOAVAILABLE | | |
|---|---|---|---|---|
| | TESTOSTERONE | TESTOSTERONE | ESTRADIOL | DHT |
| BASELINE | 6,290 pg/mL | 843 pg/mL | 47 pg/mL | 42 ng/dL |
| (DAY 0) | | | | |
| DAY 7 | 6,400 pg/mL | 966 pg/mL | 28 pg/mL | 36 ng/dL |
| DAY 14 | 7,090 pg/mL | 1,014 pg/mL | 23 pg/mL | 49 ng/dL |

These data demonstrate a 13% increase in plasma total testosterone, a 20% increase in plasma bioavailable testosterone, and a 51% reduction in plasma estradiol in only 14 days of Kneller's Trione with daily dosing at 25 mg. The starting testosterone-to-estradiol ratio was 133.83 to 1 and the ending testosterone-to-estradiol ratio was 308.26 to 1, representing a change of 130%.

The second study subject was a man, aged 21, who was dosed once per day orally with 50 mg per day of Kneller's Trione. Table 2 summarizes the data obtained.

TABLE 2
50 mg per day, single dose, Kneller's Trione

| | TOTAL | BIOAVAILABLE | | |
|---|---|---|---|---|
| | TESTOSTERONE | TESTOSTERONE | ESTRADIOL | DHT |
| BASELINE | 8,570 pg/mL | 169.7 pg/mL | 36 pg/mL | 47 ng/dL |
| (DAY 0) | | | | |
| DAY 7 | 11,000 pg/mL | 209 pg/mL | 35 pg/mL | 46 ng/dL |
| DAY 14 | 11,000 pg/mL | 209 pg/mL | 36 pg/mL | 49 ng/dL |

These data reveal a 28% increase in plasma total testosterone, a 21% increase in plasma bioavailable testosterone, and no change in plasma estradiol in only 14 days of Kneller's Trione daily dosing at 50 mg. The starting testosterone-to-estradiol ratio was 238.06 to 1 and the ending testosterone-to-estradiol ratio was 305.56 to 1, representing a change of 28%.

The third study subject was a man, aged 22, who was dosed once per day orally with 100 mg per day of Kneller's Trione. Table 3 summarizes the data obtained.

TABLE 3
100 mg per day, single dose, Kneller's Trione

| | TOTAL | BIOAVAILABLE | | |
|---|---|---|---|---|
| | TESTOSTERONE | TESTOSTERONE | ESTRADIOL | DHT |
| BASELINE | 4,700 pg/mL | 794 pg/mL | 46 pg/mL | 35 ng/dL |
| (DAY 0) | | | | |
| DAY 7 | 7,910 pg/mL | 1,432 pg/mL | 44 pg/mL | 48 ng/dL |
| DAY 14 | 9,050 pg/mL | 1,957 pg/mL | 34 pg/mL | 41 ng/dL |

These data show a 93% increase in plasma total testosterone, a 146% increase in plasma bioavailable testosterone, and a 26% reduction in plasma estradiol in only 14 days of Kneller's Trione daily dosing at 100 mg. The starting testosterone-to-estradiol ratio was 102.17 to 1 and the ending testosterone-to-estradiol ratio was 266.18 to 1, representing a change of 161%.

Although dosing of Kneller's Trione went as high as 750 mg per day in this pilot study, no added benefits were seen with dosages this high. Dosages of 250 mg and 500 mg per day also raised total testosterone and bioavailable testosterone levels substantially and safely but did not seem to offer any added benefit over a dose of 100 mg per day. At the 750 mg per day oral dosing level, occurrence of a priaprism caused the subject to withdraw from participation in this pilot study. No other adverse events were noted by any study subject during this pilot study. Observations of every subject's liver function, kidney function, blood lipid levels, blood pressure, heart rate, respirations, and body temperature were made at frequent intervals during this pilot study and were found to be within medically established normal ranges and values.

What is claimed is:

1. A method of increasing endogenous testosterone production in a male comprising administering an effective amount of 5α-androstane-3,6,17-trione (Kneller's Trione) to said male.

2. A method of claim 1 wherein the increase in endogenous testosterone production results in an absolute increase in the testosterone-to-estrogen ratio (T/E).

3. A method of claim 1 wherein 5α-androstane-3,6,17-trione (Kneller's Trione) is administered orally, transdermally, parenterally, buccally, sublingually, or rectally.

4. A method of claim 1 wherein the effective amount of 5α-androstane-3,6,17-trione (Kneller's Trione) is from about 25 mg to about 750 mg per day.

5. A method of claim 2 wherein the effective dose of 5α-androstane-3,6,17-trione (Kneller's Trione) is from about 25 mg to about 750 mg per day.

6. A method of claim 1 wherein the increase in endogenous testosterone induces an increase in muscle mass.

7. A method of claim 2 wherein the increase in T/E ratio induces an increase in muscle mass.

8. A method of claim 4 whereby the increase in testosterone induces an increase in muscle mass.

9. A method of claim 5 wherein the increase in T/E ratio induces an increase in muscle mass.

10. A method of claim 1 wherein the increase in testosterone induces an increase in athletic performance.

11. A method of claim 2 wherein the increase in T/E ratio induces an increase in athletic performance.

12. A method of claim 4 wherein the increase in testosterone induces an increase in athletic performance.

13. A method of claim 5 wherein the increase in T/E ratio induces an increase in athletic performance.

14. A method of claim 1 wherein the increase in testosterone induces an increase in libido.

15. A method of claim 2 wherein the increase in T/E ratio induces an increase in libido.

16. A method of claim 4 wherein the increase in testosterone induces an increase in libido.

17. A method of claim 5 wherein the increase in T/E ratio induces an increase in libido.

18. A formulation comprising 5α-androstane-3,6,17-trione (Kneller's Trione) in an amount effective to increase endogenous testosterone production in a male to whom the formulation is administered.

19. A formulation of claim 18 wherein the formulation comprises 5α-androstane-3,6,17-trione (Kneller's Trione) in an amount sufficient to deliver from about 25 to about 750 mg per day to a user.

20. A formulation of claim 18 wherein the formulation is suitable for oral delivery.

21. A formulation of claim 18 wherein the formulation is a dietary supplement.

22. A formulation of claim 18 wherein the formulation comprises from about 2.5% to about 100% 5α-androstane-3,6,17-trione (Kneller's Trione).

23. A formulation of claim 18 wherein the formulation comprises from about 10% to about 100% 5α-androstane-3,6,17-trione (Kneller's Trione).

24. A formulation of claim 18 wherein the formulation comprises from about 25% to about 100% 5α-androstane-3,6,17-trione (Kneller's Trione).

25. A formulation of claim 18 wherein the formulation comprises from about 50% to about 100% 5α-androstane-3,6,17-trione (Kneller's Trione).
